(19) United States
(12) Patent Application Publication
(10) Pub. No.: US 2010/0260692 A1
(43) Pub. Date: Oct. 14, 2010

(54) MOUTHWASH COMPOSITION COMPRISING XANTHAN GUM AND SODIUM FLUORIDE

(76) Inventor: Frank LIPPERT, Weybridge, Surrey, (GB)

Correspondence Address: GlaxoSmithKline;GLOBAL PATENTS -US, UW2220, P. O. BOX 1539, KING OF PRUSSIA, PA, 19406-0939, US

(21) Appl. No.: 12/665,648

(22) PCT Filed: Jun. 18, 2008
(86) PCT No.: PCT/EP2008/057677

§ 371 Date: Jun. 14, 2010

(30) Foreign Application Priority Data
Jun. 21, 2007 (GB) 0712113.0

(51) Int. Cl. A61K 8/21; A61Q 11/00

(52) U.S. Cl. 424/52

(57) ABSTRACT

An acidic mouthwash is described comprising xanthan gum and an alkali metal fluoride which enhances fluoride uptake into teeth and provides protection against acidic challenges.

### FIELD OF THE INVENTION

The present invention relates to an acidic oral care mouthwash composition comprising xanthan gum and a source of fluoride ions. The presence of xanthan gum in such an oral composition enhances fluoride uptake into teeth, thereby strengthening and hardening dental enamel, and providing protection against acidic challenges.

Such compositions are of use in providing protection against dental caries. They are also of use in combating (i.e. helping to prevent, inhibit and/or treat) dental erosion and/or tooth wear.

### BACKGROUND OF THE INVENTION

Tooth mineral is composed predominantly of calcium hydroxyapatite, Ca₁₀(PO₄)₆(OH)₂, which may be partially substituted with anions such as carbonate or fluoride, and cations such as zinc or magnesium. Tooth mineral may also contain non-apatitic mineral phases such as octacalcium phosphate and calcium carbonate.

Tooth loss may occur as a result of dental caries, which is a multifactorial disease where bacterial acids such as lactic acid produce sub-surface demineralisation that does not fully remineralise, resulting in progressive tissue loss and eventually cavity formation. The presence of a plaque biofilm is a prerequisite for dental caries, and acidogenic bacteria such as Streptococcus mutans may become pathogenic when levels of easily fermentable carbohydrate, such as sucrose, are elevated for extended periods of time.

Even in the absence of disease, loss of dental hard tissues can occur as a result of acid erosion and/or physical tooth wear; these processes are believed to act synergistically. Exposure of the dental hard tissues to acid causes demineralisation, resulting in surface softening and a decrease in mineral density. Under normal physiological conditions, demineralised tissues self-repair through the remineralising effects of saliva. Saliva is supersaturated with respect to calcium and phosphate, and in healthy individuals saliva secretion serves to wash out the acid challenge, and raises the pH so as to alter the equilibrium in favour of mineral deposition.

Dental erosion (i.e. acid erosion or acid wear) is a surface phenomenon that involves demineralisation, and ultimately complete dissolution of the tooth surface by acids that are not of bacterial origin. Most commonly the acid will be of dietary origin, such as citric acid from fruit or carbonated drinks, phosphoric acid from cola drinks and acetic acid such as from vinaigrette. Dental erosion may also be caused by repeated contact with hydrochloric acid (HCl) produced by the stomach, which may enter the oral cavity through an involuntary response such as gastroesophageal reflux, or through an induced response as may be encountered in sufferers of bulimia.

Tooth wear (i.e. physical tooth wear) is caused by attrition and/or abrasion. Attrition occurs when tooth surfaces rub against each other, a form of two-body wear. An often dramatic example is that observed in subjects with bruxism, a grinding habit where the applied forces are high, and is characterised by accelerated wear, particularly on the occlusal surfaces. Abrasion typically occurs as a result of three-body wear and the most common example is that associated with brushing with a toothpaste. In the case of fully mineralised enamel, levels of wear caused by commercially available toothpastes are minimal and of little or no clinical consequence. However, if enamel has been demineralised and softened by exposure to an erosive challenge, the enamel becomes more susceptible to tooth wear. Dentine is much softer than enamel and consequently is more susceptible to wear. Subjects with exposed dentine should avoid the use of highly abrasive toothpastes, such as those based on alumina. Again, softening of dentine by an erosive challenge will increase susceptibility of the tissue to wear.

Dentine is a vital tissue that in vivo is normally covered by enamel or cementum depending on the location i.e. crown versus root respectively. Dentine has a much higher organic content than enamel and its structure is characterised by the presence of fluid-filled tubules that run from the surface of the dentine-enamel or dentine-cementum junction to the odontoblast/pulp interface. It is widely accepted that the origins of dentine hypersensitivity relate to changes in fluid flow in exposed tubules, (the hydrodynamic theory), that result in stimulation of mechanoreceptors thought to be located close to the odontoblast/pulp interface. Not all exposed dentine is sensitive since it is generally covered with a smear layer; an occlusive mixture comprised predominantly of mineral and proteins derived from dentine itself, but also containing organic components from saliva. Over time, the lumen of the tubule may become progressively occluded with mineralised tissue. The formation of reparative dentine in response to trauma or chemical irritation of the pulp is also well documented. Nonetheless, an erosive challenge can remove the smear layer and tubule “plugs” causing outward dentinal fluid flow, making the dentine much more susceptible to external stimuli such as hot, cold and pressure. As previously indicated, an erosive challenge can also make the dentine surface much more susceptible to wear. In addition, dentine hypersensitivity worsens as the diameter of the exposed tubules increases, and since the tubule diameter increases as one proceeds in the direction of the odontoblast/pulp interface, progressive dentine wear can result in an increase in hypersensitivity, especially in cases where dentine wear is rapid.

Loss of the protective enamel layer through erosion and/or acid-mediated wear will expose the underlying dentine, and are therefore primary aetiological factors in the development of dentine hypersensitivity.

It has been claimed that an increased intake of dietary acids, and a move away from formalised meal times, has been accompanied by a rise in the incidence of dental erosion and tooth wear. In view of this, oral care compositions which can help prevent dental erosion and tooth wear and which provide protection from dental caries would be advantageous.

Oral care compositions often contain a source of fluoride ions for promoting remineralisation of teeth and for increasing the acid resistance of dental hard tissues. To be effective the fluoride ions must be available for uptake into the dental hard tissues being treated.

WO 2000/13531 (SmithKline Beecham) describes the use of viscosity modifying polymers as tooth erosion inhibitors in acidic compositions including mouthwashes, in which the pH of the compositions is less than or equal to pH 4.5. Examples of viscosity modifying polymers include polysaccharides such as alginates, xanthans and pectins. There is no disclosure of a mouthwash composition comprising such polymers in combination with a source of fluoride ions.

WO 2004/054529 (Procter & Gamble) describes a method of protecting teeth against dental erosion comprising administering an oral care composition comprising a polymeric mineral surface active agent (such as a polyphosphate, polyphosphonate or polycarboxylate) and/or a source of metal ions selected from stannous, zinc and copper, and optionally together with a source of fluoride ions. It is stated that the pH of such compositions can be within the range of about 4 to about 10, preferably from about 4.5 to about 8, and more preferably from about 5.5 to about 7. There is no suggestion that any of the claimed polymers can enhance fluoride uptake from an oral composition.

WO 2004/054531 (Procter & Gamble) describes a method of enhancing fluoridation and mineralization of teeth by administering an oral care composition comprising specialized phosphonate containing polymers together with a source of fluoride ions. It is stated that the pH of such compositions can be within the range of about 4 to about 10, preferably from about 4.5 to about 8, and more preferably from about 5.5 to about 7.

U.S. Pat. No. 4,540,576 (Johnson & Johnson) describes a neutral topical sodium fluoride gel having a pH in the range of about 6 to about 8 and containing as thickener a mixture of xanthan gum and a soluble salt of an acrylic acid polymer.

FR-A-2755010 describes the use of an oral care composition comprising a combination of a fluoride, a carboxylated vinyl polymer and a xanthan gum, to enhance the efficacy of the fluoride. There is no suggestion that the pH of such a composition should not be greater than 5.0.

WO 01/66074 (Colgate) describes a dual component dentifrice, one phase being alkaline and containing fluoride ions, the other phase being acidic and containing phosphate ions, which on mixing prior to use, provides an acidic phosphate fluoride composition (pH 4 to 6). It is suggested that the delivery of the dentifrice at an acidic pH can enhance the uptake of the fluoride ions into the tooth enamel. Such dentifrices can be thickened with various organic thickeners including natural or synthetic gums including xanthan gum or sodium carboxymethylcellulose. There is no suggestion that such thickeners can enhance the uptake of fluoride.

WO 04/012693 (Colgate) describes a dental composition having heightened desensitisation as well as heightened tooth fluoridation and remineralisation which is apparently achieved by combining a fluoride ion with a potassium salt, the composition having a pH in the range 7.5 to 9 and being buffered with an alkali metal phosphate salt.

WO 2004/054530 (Colgate) describes a method for optimizing fluoride uptake by applying a liquid dentifrice having a moderate viscosity, which may be thickened with various agents including xanthan gum and a thickening silica. In comparison with a standard dentifrice having a higher viscosity, the liquid dentifrice is described to provide more effective fluoride delivery. There is no suggestion that the pH of the liquid dentifrice should not be greater than 5.0.

WO 2006/013081 (Glaxo Group Ltd) describes oral care compositions for treating xerostomia (dry mouth) comprising a polyvinyl pyrrolidone or derivative thereof, an anionic mucoadhesive polymer such as a cellulose gum, a saccharide gum or a polyacrylic acid, and optionally a source of fluoride ions. It is stated that such compositions will have a pH which is orally acceptable, typically ranging from about pH 5 to 10 and more preferably pH 5.5 to 8. There is no suggestion that such compositions may enhance the uptake of fluoride.

The present invention is based on the discovery that incorporation of up to 0.1% by weight of xanthan gum into an oral care mouthwash composition comprising an alkali metal fluoride advantageously enhances the uptake of fluoride ions into dental enamel when the pH of the mouthwash composition is not greater than 5.0.

The beneficial effects of up to 0.1% by weight of the xanthan gum on fluoride uptake are not observed in mouthwash compositions having a pH greater than 5.0. Furthermore a mouthwash composition comprising xanthan gum in the absence of a source of fluoride ions does not appear to offer any protection against a subsequent erosive acidic challenge, suggesting that the xanthan gum is not acting to provide a surface protective coating, contrary to WO 2004/054529 which suggests that various polymers can coat and thereby protect teeth against erosion.

### SUMMARY OF THE INVENTION

Accordingly, the present invention provides an oral care mouthwash composition comprising from 0.001 to 0.1% by weight of xanthan gum and an alkali metal fluoride and having a pH not greater than 5.0.

Such compositions are of use in enhancing fluoride uptake into teeth and to provide protection against acidic challenges.

Such compositions are of use in providing protection against caries.

Such compositions are of use in combating dental erosion and/or tooth wear.

### DETAILED DESCRIPTION OF THE INVENTION

The xanthan gum may be present in an amount of 0.001 to 0.1% by weight of the total composition, for example from 0.005 to 0.05% or from 0.01 to 0.02% by weight of the total composition.

Examples of an alkali metal fluoride include sodium or potassium fluoride, in an amount to provide up to 12500 ppm of fluoride ions, suitably from 25 to 3500pm of fluoride ions, eg from 100 to 1500 ppm.

Suitably the alkali metal fluoride is sodium fluoride, in an amount to provide from 100 to 1500 ppm, for example from 200 to 500 ppm of fluoride ions.

Suitably the pH of the oral care composition is from 3.0 to 5.0, typically from 4.0 to 5.0, for example from 4.0 to 4.6, and may be adjusted by the inclusion of a pH modifying agent such as an acid (eg benzoic or hydrochloric acid) or acidic buffer (eg benzoic acid/sodium benzoate buffer).

Compositions of the present invention may further comprise a desensitising agent for combating dentine hypersensitivity. Examples of desensitising agents include a tubule blocking agent or a nerve desensitising agent and mixtures thereof, for example as described in WO 02/15809. Suitable desensitising agents include a strontium salt such as strontium chloride, strontium acetate or strontium nitrate or a potassium salt such as potassium citrate, potassium chloride, potassium bicarbonate, potassium gluconate and especially potassium nitrate.

A desensitising amount of a potassium salt is generally between 2 to 8% by weight of the total composition, for example 5% by weight of potassium nitrate can be used.

Compositions of the present invention may contain appropriate formulating agents such as thickening agents, surfactants, humectants, flavouring agents, sweetening agents, opacifying or colouring agents, preservatives and water, selected from those conventionally used in the oral care composition art for such purposes.

As xanthan gum can act as a thickening agent, suitably it is present as the sole thickening agent in compositions of the present invention.

Suitable humectants for use in compositions of the invention include glycerine, sorbitol, xylitol, isomalt, propylene glycol or polyethylene glycol, or mixtures thereof which humectant may be present in the range from 5 to 70%.

Suitable surfactants for use in the invention include polyethyleneglycols (PEG), hydrogenated caster oils, sorbitan esters, or polyethylene-polypropylene tri-block copolymers (such as Poloxamers™)

Compositions of use in the present invention may be prepared by admixing the ingredients in the appropriate relative amounts in any order that is convenient and if necessary adjusting the pH to give the desired value.

The present invention also provides a method for enhancing fluoride uptake into teeth and to provide protection against acidic challenges which comprises applying an effective amount of a composition as hereinbefore defined to an individual in need thereof.

The Invention is Further Illustrated by the Following Examples and Comparative Examples Which were Tested in the Following Studies Assessing Fluoride Efficacy.

Study 1

### Enamel Fluoride Uptake (EFU)

The purpose of this in vitro study was to determine the effect of 6 mouthwashes on promoting fluoride uptake into incipient enamel lesions. The mouthwash formulation details were as follows:

TABLE 1

| | Ex 1 | Ex A | Ex B | Ex C | Ex D | Ex E |
|---|---|---|---|---|---|---|
| Ingredient Name | % w/w | % w/w | % w/w | % w/w | % w/w | % w/w |
| purified water | 85.13 | 85.14 | 85.13 | 85.14 | 85.13 | 85.14 |
| glycerin | 7.00 | 7.00 | 7.00 | 7.00 | 7.00 | 7.00 |
| sorbitol | 5.00 | 5.00 | 5.00 | 5.00 | 5.00 | 5.00 |
| pluronic F 108 | 1.00 | 1.00 | 1.00 | 1.00 | 1.00 | 1.00 |
| cremaphor RH 60 | 1.00 | 1.00 | 1.00 | 1.00 | 1.00 | 1.00 |
| sodium benzoate | 0.50 | 0.50 | 0.50 | 0.50 | 0.50 | 0.50 |
| cetylpyridinium chloride | 0.05 | 0.05 | 0.05 | 0.05 | 0.05 | 0.05 |
| methyl paraben | 0.10 | 0.10 | 0.10 | 0.10 | 0.10 | 0.10 |
| propyl paraben | 0.10 | 0.10 | 0.10 | 0.10 | 0.10 | 0.10 |
| sodium saccharin | 0.05 | 0.05 | 0.05 | 0.05 | 0.05 | 0.05 |
| sodium fluoride | 0.0553 | 0.0553 | 0.0553 | 0.0553 | 0.0553 | 0.0553 |
| xanthan gum | 0.01 | — | 0.01 | — | 0.01 | — |
| disodium phosphate | — | — | — | — | 0.0027 | 0.0027 |
| anhydrous | | | | | | |
| monosodium phosphate | — | — | — | — | 0.00156 | 0.00156 |
| anhydrous | | | | | | |
| pH (adjusted with | 4.5 | 4.5 | 5.5 | 5.5 | 7.0 | 7.0 |
| HCl or NaOH) | | | | | | |

Example 1 describes a mouthwash composition of the present invention having a pH of 4.5, and comprising xanthan gum and sodium fluoride as the fluoride source. Comparative Examples A to E fall outside the scope of the present invention either having no xanthan gum and/or a pH greater than 5.

The test procedure was a modification of Test Method #40 in the FDA Monograph including the formation of a caries-like (subsurface) lesion that is formed using a solution of 0.1 M lactic acid and 0.2% Carbopol 907, 50% saturated with hydroxyapatite (HAP) at a pH of 5.0.

### Specimen Preparation

Sound human teeth were selected and cleaned of all adhering soft tissue. A core of enamel 3 mm in diameter was prepared from each tooth by cutting perpendicularly to the labial surface with a hollow-core diamond drill bit. This was performed under water to prevent overheating of the specimens. Each specimen was embedded in the end of an acrylic rod (¼″ diameter×2″ long) using methyl methacrylate. The excess acrylic was cut away exposing the enamel surface. The enamel specimens were ground with 600 grit wet/dry paper for 10 minutes and then polished with micro-fine Gamma Alumina 45 minutes. The resulting specimen was a 3 mm in diameter disk all covered with the acrylic, except the exposed enamel surface.

The enamel specimens were visually inspected for cracks, exposed dentin and lesions using a 10× magnifying glass. Specimens containing any of these imperfections were rejected. Twelve specimens per group were used for this study. Specimens were numbered randomly and assigned to groups by their number in sequential order (1-12 in group 1, 13-24 in group 2, etc.)

Pre-Treatment (indigenous) Fluoride Determination

Each enamel specimen was demineralised into 0.5 ml of 1 M HClO₄ for 15 seconds. Throughout this period the demineralization solution was continuously agitated with an up and down motion of the specimens. Immediately after the demineralization, the specimens were rinsed thoroughly with deionised water. A sample of each solution was then buffered with total ionic strength buffer (TISAB) II (0.25 ml sample, 0.5 ml TISAB II and 0.25 ml 1N NaOH) and the fluoride content determined by comparison to a similarly prepared standard curve (1 ml standard+1 ml TISAB II). To calculate the amount of enamel removed by the 15 seconds demineralization procedure, the calcium content of the demineralization solution was determined by atomic absorption (0.05 ml sample, 1 ml LaCl₃ and 4.95 ml deionised water). From these data, the fluoride level of each specimen prior to treatment (indigenous) was calculated.

### Creation of Subsurface Caries-Like Lesion

To remove the demineralised layer, specimens were ground again but for 10 seconds and polished for 45 minutes, as described above. An incipient, caries-like lesion was formed in each specimen by placing them into a 0.1 M lactic acid/0.2% Carbopol 907/HAP solution for 24 hours, at 37° C. After this demineralization period, specimens were rinsed thoroughly with deionised water and stored at 100% relative humidity until use.

### Treatments

The specimens were immersed into 25 ml of their assigned mouthwash with constant stirring (100 rpm) for 30 minutes, at room temperature. Following treatment, specimens were rinsed thoroughly with deionised water.

### Post-Treatment Fluoride Determination

Specimens were demineralised again into 0.5 ml of 1 M HClO₄ for 15 seconds and the resulting solutions were analyzed for fluoride and calcium content, as described above. From these data, the fluoride level in each specimen after treatment was calculated.

### Data Management and Analysis

The pre-treatment fluoride (indigenous) level of each specimen was subtracted from the post treatment level to determine the amount of fluoride acquired (fluoride uptake) by the enamel due to the test treatment.

Data was analyzed using a one-way analysis of variance model [Sigma Stat (3.0) Software]. Data were further analyzed using all pairwise comparisons (Student-Newman-Keuls method). All analyses were done with the significance level set at below 0.05.

### Results

The results are shown in Table 2 and Graph 1 below.

TABLE 2

| | formulation | EFU | SEM |
|---|---|---|---|
| Ex 1 | pH 4.5 XG | 9454 | 613 |
| Ex A | pH 4.5 | 6819 | 373 |
| Ex B | pH 5.5 XG | 2082 | 86 |
| Ex C | pH 5.5 | 2151 | 86 |
| Ex D | pH 7.0 XG | 1099 | 51 |
| Ex E | pH 7.0 | 1085 | 29 |

(SEM—standard error of the mean)

Treatment with Example 1 (pH4.5 XG) did result in a statistically significantly higher EFU than Example A, the same formulation excluding xanthan gum (pH4.5), as well as compared to Examples B to E. Referring to Graph 1 it is evident that xanthan gum in a mouthwash formulation at pH 5.5 and pH 7.0 did not lead to a statistically significant improvement in EFU.

Study 2

### Microindentation as a Measure of Enamel Hardness in Assessing Fluoride Efficacy Against a Cariogenic Challenge

A simple in vitro model was developed to investigate the fluoride efficacy of mouthwash formulations (Example 1 and Comparative Examples A to E) to protect against a cariogenic challenge. Polished bovine enamel samples were treated with a mouthwash for 2 min, immediately followed by a 5 hour treatment with a lactic acid buffer under static conditions. The composition of the lactic acid buffer can be found in Table 3 below:

TABLE 3

| | | |
|---|---|---|
| Ingredient name | concentration | g/kg |

| calcium chloride dihydrate | 3 | mM | 0.441 |
|---|---|---|---|
| potassium phosphate monobasic | 1.8 | mM | 0.245 |
| lactic acid (30% w/w) | 0.1 | M | 30.027 |
| Carboxymethylcellulose (high | 1% | | 10.000 |
| viscosity) | | | |
| sodium azide | 3.08 | mM | 0.200 |

| | | |
|---|---|---|
| purified water | — | 959.100 |

| | |
|---|---|
| potassium hydroxide | (used for pH 4.5 adjustment) |

Changes in surface microhardness (SMH) before and after treatments were used to determine possible caries-protection effects of the mouthwash treatment.

Polished bovine enamel samples were prepared by cutting bovine teeth into approx. 2×2 mm pieces which were embedded in epoxy resin (Stycast 1266, Hitek) and polished to mirror flatness on a lapping machine using silicone carbide discs up to 4000 grit (Kemet). Approx. 15-25 samples were prepared per tooth. Samples were stored in tap water prior to investigation.

For each experiment, enamel samples were measured for baseline surface microhardness (VHN—Vickers Hardness Number) employing a Vickers microhardness tester (Struers). Six indentations were performed per sample applying a load of 1.961 N with a holding period of 15 s. Samples were then randomized into treatment groups based on their average baseline VHN.

For the mouthwash treatment, one 5 ml beaker containing 4 ml of the mouthwash formulation to be tested was prepared per sample. The samples were placed into their beakers for 2 min. Samples were rinsed with DI (deionised) water and immediately processed further.

For the cariogenic challenge, one 10 ml beaker containing 10 ml lactic acid buffer was prepared per sample. The samples were placed into their beakers and left unstirred for 5 h. Samples were then rinsed with DI water and left to dry.

Both mouthwash and lactic acid buffer treatments were carried out under ambient conditions.

Samples were then measured again for SMH as described above. A one-way ANOVA at the 95% confidence level was employed for each experiment. A multiple range test (Fisher's least significant difference procedure) at a 95% confidence level was performed to identify statistically homogeneous groups.

Using the microindentation technique the following results were obtained (Table 4; Graph 2; “untreated”×baseline VHN; “demin”×VHN after cariogenic challenge):

TABLE 4

| | | VHN | | VHN SD | |
|---|---|---|---|---|---|
| | formulation | untreated | demin | untreated | demin |
| Ex 1 | pH 4.5 XG | 353 | 205 | 14 | 24 |
| Ex A | pH 4.5 | 353 | 186 | 16 | 13 |
| Ex B | pH 5.5 XG | 352 | 157 | 17 | 13 |
| Ex C | pH 5.5 | 352 | 157 | 12 | 10 |
| Ex D | pH 7.0 XG | 352 | 154 | 11 | 7 |
| Ex E | pH 7.0 | 352 | 140 | 11 | 13 |

(SD—Standard Deviation)

Treatment with Example 1 (pH4.5 XG) did result in a statistically significantly better demineralisation protection than Example A, the same formulation excluding xanthan gum (pH4.5), as well as compared to Examples B to E. Referring to Table 4 and Graph 2 it is also evident that xanthan gum in a mouthwash formulation at pH 5.5 and pH 7.0 did not lead to a statistically significant improvement in demineralisation protection.

Study 3

### Microindentation as a Measure of Enamel Hardness in Assessing Fluoride Efficacy Against an Erosive Challenge

A simple in vitro model was developed to investigate the fluoride efficacy of mouthwash formulations to protect against dental erosion. Polished bovine enamel samples were treated with a mouthwash for 2 min, immediately followed by a 20-min treatment with artificial orange juice (1% citric acid, pH 3.75) under static conditions. Changes in surface microhardness before and after treatments were used to determine possible erosion-protection effects of the mouthwash treatment.

Polished bovine enamel samples were prepared by cutting bovine teeth into approx. 2×2 mm pieces which were embedded in epoxy resin (Stycast 1266, Hitek) and polished to mirror flatness on a lapping machine using silicone carbide discs up to 4000 grit (Kemet).

Approx. 15-25 samples were prepared per tooth. Samples were stored in tap water prior to investigation.

For each experiment, enamel samples were measured for baseline surface microhardness (VHN) employing a Vickers microhardness tester (Struers). Six indentations were performed per sample applying a load of 1.961 N with a holding period of 15 s. Samples were then randomized into treatment groups based on their average baseline VHN.

For the mouthwash treatment, one 5 ml beaker containing 4 ml of the mouthwash formulation to be tested was prepared per sample. The samples were placed into their beakers for 2 min. Samples were rinsed with DI water and immediately processed further.

For the artificial orange juice (1% citric acid monohydrate in DI water, adjusted to pH 3.75 with KOH) treatment, one 10 ml beaker containing 10 ml artificial orange juice was prepared per sample. The samples were placed into their beakers and left unstirred for 20 min. Samples were then rinsed with DI water and left to dry.

Both mouthwash and artificial orange juice treatments were carried out under ambient conditions.

Samples were then measured again for SMH as described above. A one-way ANOVA at the 95% confidence level was employed for each experiment. A multiple range test (Fisher's least significant difference procedure) at a 95% confidence level was performed to identify statistically homogeneous groups.

Formulation details of the tested mouthwash formulations can be found in Table 5 below:

TABLE 5

| | Ex 2 | Ex 3 | Ex 4 | Ex F | Ex G | Ex H | Ex I | Ex J |
|---|---|---|---|---|---|---|---|---|
| Ingredient Name | % w/v | % w/v | % w/v | % w/v | % w/v | % w/v | % w/v | % w/v |
| purified water | 85.35 | 85.31 | 85.26 | 85.45 | 85.40 | 85.36 | 85.31 | 85.31 |
| glycerin | 7.00 | 7.00 | 7.00 | 7.00 | 7.00 | 7.00 | 7.00 | 7.00 |
| sorbitol | 5.00 | 5.00 | 5.00 | 5.00 | 5.00 | 5.00 | 5.00 | 5.00 |
| pluronic F 108 | 1.00 | 1.00 | 1.00 | 1.00 | 1.00 | 1.00 | 1.00 | 1.00 |
| cremophor RH 60 | 1.00 | 1.00 | 1.00 | 1.00 | 1.00 | 1.00 | 1.00 | 1.00 |
| sodium benzoate | 0.50 | 0.50 | 0.50 | 0.50 | 0.50 | 0.50 | 0.50 | 0.50 |
| sodium saccharin | 0.05 | 0.05 | 0.05 | 0.05 | 0.05 | 0.05 | 0.05 | 0.05 |
| xanthan gum | 0.01 | 0.05 | 0.10 | — | 0.05 | — | 0.05 | 0.05 |
| sodium fluoride | 0.088 | 0.088 | 0.088 | — | — | 0.088 | 0.088 | 0.088 |
| pH (adjusted with | 4.5 | 4.5 | 4.5 | 4.5 | 4.5 | 4.5 | 5.5 | 6.5 |
| HCl or NaOH) | | | | | | | | |

Examples 2 to 4 describe mouthwash compositions of the present invention having a pH of 4.5, and comprising xanthan gum and sodium fluoride as the fluoride source.

These examples were compared with Examples F to J (falling outside the scope of the present invention either having no fluoride ion source and/or no xanthan gum and/or a pH greater than 5) to assess their ability to protect dental enamel against an erosive challenge.

### Effect of pH and Xanthan Gum on Fluoride Efficacy

Using the microindentation technique the following results were obtained. Table 6 and Graph 3 describe the effect of pH and xanthan gum on the ability of fluoride containing mouthwashes to protect against an acidic erosive challenge.

TABLE 6

| | | VHN | | VHN SD | |
|---|---|---|---|---|---|
| | formulation | untreated | demin | untreated | demin |
| Ex 3 | pH 4.5 + F + XG | 375 | 284 | 19 | 12 |
| Ex F | pH 4.5 − F − XG | 374 | 236 | 18 | 6 |
| Ex G | pH 4.5 − F + XG | 374 | 221 | 17 | 23 |
| Ex H | pH 4.5 + F − XG | 375 | 263 | 19 | 12 |
| Ex I | pH 5.5 + F + XG | 374 | 259 | 18 | 14 |
| Ex J | pH 6.5 + F + XG | 374 | 251 | 16 | 15 |

(SD—Standard Deviation)

Referring to Graph 3 it is evident that xanthan gum on its own in the absence of fluoride in a mouthwash formulation at pH 4.5 (Example G: pH4.5−F+XG) offered no protection against a subsequent erosive challenge and appeared to be directionally less effective than the same formulation excluding xanthan gum (Example F: pH4.5−F−XG). By contrast the combination of fluoride and xanthan gum at pH 4.5 (Example 3: pH4.5+F+XG) offered statistically significantly greater protection against subsequent erosion than fluoride on its own (Example H: pH4.5+F−XG). An increase in pH from 4.5 to 5.5 or 6.5 of the xanthan gum-fluoride mouthwash formulation resulted in a loss in efficacy (Example I: pH5.5+F+XG and Example J: pH6.5+F+XG).

### Effect of Xanthan Gum Concentration on Fluoride Efficacy

Table 7 and Graph 4 describe the results of formulations (Examples 2 to 4) prepared to investigate the effect of xanthan gum concentration when formulated at pH 4.5 with 400 ppm fluoride to protect against an acidic erosive challenge.

TABLE 7

| | | VHN | | VHN SD | |
|---|---|---|---|---|---|
| | formulation | untreated | demin | untreated | demin |
| Ex 2 | 0.01% XG | 376 | 291 | 21 | 14 |
| Ex 3 | 0.05% XG | 375 | 294 | 13 | 15 |
| Ex 4 | 0.10% XG | 376 | 306 | 18 | 11 |

(SD—Standard Deviation)

Referring to Table 7 and Graph 4, there was no statistically significant difference between any formulation, but directional differences can be seen, clearly favouring a higher xanthan gum concentration (Examples 2, 3 and 4 respectively relate to “0.01% XG”, “0.05% XG”, “0.1% XG” referred to in FIG. 4).

1. An oral care mouthwash composition comprising from 0.001 to 0.1% by weight of xanthan gum and an alkali metal fluoride and having a pH not greater than 5.0.

2. A composition according to claim 1 wherein xanthan gum is present in an amount of 0.005 to 0.05% by weight of the total composition.

3. A composition according to claim 1 wherein the alkali metal fluoride is sodium or potassium fluoride.

4. A composition according to claim 3 wherein the alkali metal fluoride is sodium fluoride in an amount which provides from 100 to 1500 ppm of fluoride ions.

5. A composition according to claim 1 having a pH from 4.0 to 5.0.

6. A composition according to claim 1 further comprising a desensitizing agent.

7. A method for enhancing fluoride uptake into teeth and to provide protection against acidic challenges which comprises applying an effective amount of a composition as defined in claim 1 to an individual in need thereof.
